(19) United States
(12) Patent Application Publication
(10) Pub. No.: US 2004/0219669 A1
(43) Pub. Date: Nov. 4, 2004

(54) Method for detection of hemopoietic stem and progenitor cells

(75) Inventor: Yoshimoto Katsura, Yokohama-shi (JP)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(73) Assignee: INSTITUTE FOR APPLIED MEDICINE, LTD., Tokyo (JP)

(21) Appl. No.: 10/426,041

(22) Filed: Apr. 29, 2003

(51) Int. Cl. G01N 33/567; C12N 5/08

(52) U.S. Cl. 435/372; 435/7.21

(57) ABSTRACT

The present invention provides a method for detection of hematopoietic stem and/or progenitor cells, comprising the steps of culturing individual cells in a culture environment that supports the generation of myeloid, erythroid, T and B lineage cells and determining the developmental potential of individual cells towards these four lineages (MLP<METB> assay), and a method for detection of hematopoietic stem and/or progenitor cells, comprising the steps of culturing individual cells in a culture environment that supports the generation of myeloid, erythroid, and B lineage cells and determining the developmental potential of the individual cells towards these three lineages (MLP<MEB> assay).

### BACKGROUND OF THE INVENTION

(1) Field of the Invention

The present invention relates to a method for detecting in vitro the hemopoietic stem cells and hemopoietic progenitor cells, and a culture system therefor. The present invention is useful for studies on blood cell differentiation, and it may have applications to medical treatments such as treatment of leukemia and gene therapy.

(2) Description of the Related Art

The development of hematopoietic cells has long been investigated with different systems by different research groups. Studies on the myelo-erythroid lineage development has mainly been performed by the colony forming unit-culture (CFU-C) assay, which is effective in determining the developmental potential of individual progenitors towards the erythroid and myeloid lineages. In these studies, however, the developmental potential towards the T or B cell lineages has not been taken into consideration. T cell development is highly dependent upon the thymic microenvironment, and the growth of B cell needs stromal cells. Furthermore, in studies on T or B cell development, usually 10³ or more cells have been used in the culture, namely the assays have been performed at a population level.

With respect to the hematopoietic stem cell (HSC) (the pluripotent progenitor capable of generating all hematopoietic lineage cells), HSCs can be detected in animals such as mice by transplanting serially diluted number of cells into irradiated recipients. Such an experimental method is complicated and expensive. Also, there has been no method to detect human HSCs.

The present inventor previously developed a clonal assay, called the MLP<MTB> assay, for determining the developmental potential of individual progenitors towards myeloid, T and B cell lineages (Kawamoto et al., Int. Immunol. 9, 1011-1019 (1997). However, this method is unable to discriminate the HSCs and progenitors restricted to myeloid, T and B cell lineages (p-MTB). Therefore, it has been difficult to clarify the exact pathway of blood differentiation from the HSCs.

### SUMMARY OF THE INVENTION

In one aspect, the present invention provides a method for detection of hematopoietic stem cells and/or hematopoietic progenitor cells, comprising the steps of culturing individual cells in a culture environment that supports the generation of myeloid, erythroid, T and B lineage cells and determining the developmental potential of individual cells towards these four lineages (MLP<METB> assay).

In this method, the determination of the developmental potential is preferably made by analyzing the cells generated from a single progenitor using a flow cytometer.

In another aspect, the present invention provides a culture system for use in detecting hematopoietic stem cells and/or hematopoietic progenitor cells, which supports the generation of myeloid, erythroid, T and B lineage cells. Preferably, the culture system comprises a thymic organ culture system and further comprises cytokines that support the growth of myeloid, erythroid, and B lineage cells.

In another aspect, the present invention provides a method for detection of hematopoietic stem cells and/or hematopoietic progenitor cells, comprising the steps of culturing individual cells in a culture environment that supports the generation of myeloid, erythroid, and B lineage cells and determining the developmental potential of the individual cells towards these three lineages (MLP<MEB> assay).

In another aspect, the present invention provides a culture system for use in detecting hematopoietic stem cells and/or hematopoietic progenitor cells, which supports the generation of myeloid, erythroid, and B lineage cells.

### BRIEF DESCRIPTION OF THE DRAWINGS

FIG. 1 illustrates the preparation of murine fetal liver cells, cell staining with various antibodies, and separation of the progenitor-enriched fraction using a FACS Vantage.

FIG. 2 illustrates an example of basic experiment for development of MLP assay. In this experiment 30 Sca-1⁺ murine fetal liver cells were cultured together with a deoxyguanosine (dGuo)-treated fetal thymus (FT) lobe in the presence or absence of a cytokine cocktail indicated. Flow cytometric profiles of cells in these cultures are shown in FIGS. 2(B) and 2(C).

FIG. 3 illustrates the MLP<MTB> assay. FIG. 3(A) shows the procedures of MLP<MTB> assay. FIGS. 3(B), 3(C), and 3(D) show FACS profiles of cells generated from six different progenitors.

FIG. 4 shows FACS profiles of cells derived from four types of progenitors, which were newly identified by the MLP<METB> assay. The other five types of progenitors detected by this method are the same as shown in FIGS. 3(C) and 3(D).

FIG. 5 shows the distribution of different types of progenitors detected by the MLP<METB> assay. 390 Lin⁻ c-kit⁺ Sca-1⁺ cells from murine fetal liver were individually cultured and the numbers of different types of progenitors are plotted.

FIG. 6 shows the procedures of MLP<MEB> assay and FACS profiles of two important types of progenitors detected by this assay.

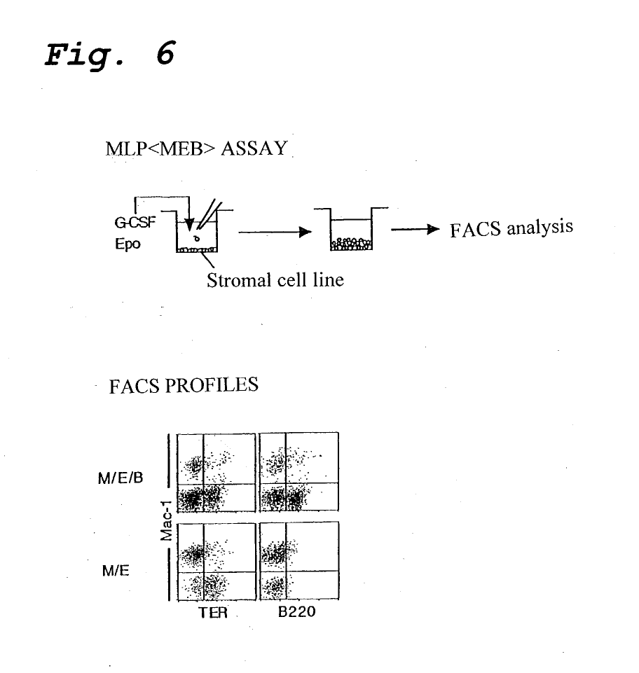

### DESCRIPTION OF THE PREFERRED EMBODIMENTS

The method of the present invention for detection of hematopoietic stem and/or progenitor cells is explained in detail below.

(1) MLP<MTB> Assay

This is an assay which was developed by the present inventor previously for examining the developmental potential of individual stem/progenitor cells towards the myeloid, T and B cell lineages. For example, single cells from Lin⁻ c-kit⁺ Sca-1⁺ population of murine fetal liver are cultured together with a deoxyguanosine (dGuo)-treated fetal thymic (FT) lobe in the presence of stem cell factor (SCF)(10 ng/ml), interleukin 3 (IL-3)(3 ng/ml) and interleukin 7 (IL-7)(5 ng/ml) in a 96-well V-bottom plate. The cells generated are harvested and analyzed. The type of progenitors seeded in each well are determined from the flow cytometric profiles of the cells generated.

This assay makes it possible to classify the stem/progenitor cells into seven types, which are a multipotential progenitor capable of generating myeloid, T and B cells (p-Multi), three types of bipotent progenitors (p-MT, p-MB and p-TB), and three types of unipotent progenitors (p-M, p-T and p-B). Among these theoretically possible seven types, the p-TB type progenitors has never been detected, although the existence of p-TB had been presumed and named as the common lymphoid progenitors.

(2) MLP<METB> Assay of the Present Invention

It remains unclear whether the multipotent progenitors (p-Multi), as determined by the above MLP<MTB> assay, represent the hematopoietic stem cells or comprise also progenitors restricted to the myeloid, T, and B cell lineages (p-MTB).

MLT<METB> assay has been devised to examine the developmental potential of a single cell towards the myeloid, erythroid, T, and B cell lineages.

In this assay, a sample containing hematopoietic stem and/or progenitor cells, such as murine fetal liver cells, murine fetal blood, murine bone marrow cells, and thymus cells may be examined.

A cell population in which stem and/or progenitor cells are enriched may be separated by using a fluorescence-activated cell sorter (FACS). Individual cells contained in the population are cultured.

The culture system used in the present method is a culture system which supports the development and growth of myeloid, erythroid, T and B cells. More specifically, a thymic organ culture system is used, and the culture medium is supplemented with cytokines that supports the growth of myeloid, erythroid and B lineage cells. Preferably, dGuo-treated murine FT is used, and the culture medium is supplemented with 5-20 ng/ml of stem cell factor (SCF), 1-5 ng/ml of interleukin 3 (IL-3), 2-10 ng/ml of interleukin 7 (IL-7), and 2-5 U/ml of erythropoietin (Epo). Cytokines to be added are not limited to those listed above, and some modifications are possible. For example, granulocyte-macrophage colony stimulating factor (GM-CSF) may be added in place of interleukin 3.

Culturing may be carried out at 37° C. under the high oxygen submersion (HOS) conditions (70% O₂, 25% N₂, and 5% CO₂).

The cells produced in the culture are stained with antibodies specific for each cell lineage, and are analyzed with a flow cytometer, the fluorescence-activated cell sorter (FACS). From FACS analysis data, the type of each progenitor can be determined.

According to the MLP<METB> assay, it is formally possible to discriminate 15 different types of progenitors, which are p-METB, p-MET, p-MEB, p-MTB, p-ETB, p-ME, p-MT, p-MB, p-ET, p-EB, p-TB, p-M, p-E, p-T and p-B (the type of progenitor cells is indicated as a combination of “p-” and the name of the lineage to be differentiated, and “M” means myeloid cell, “E” means erythrocyte, “T” means T cell, and “B” means B cell.)

As demonstrated in the Examples below, out of 15 possible types, nine types i.e., p-METB, p-MTB, p-ME, p-MT, p-MB, p-M, p-E, p-T, and p-B were detected and six possible types were not detected in murine fetal liver cells. p-METB may represent the stem cells themselves, and p-MTB may be progenitors which are common to myeloid, T and B cell lineages and which was not expected prior to the present invention. Namely, according to the method of the present invention, not only can a stem cell be detected, but the existence of the earliest progenitors to be differentiated into lymphoid lineages, i.e., p-MTB was also demonstrated.

(3) MLP<MEB> Assay

MLP<METB> assay mentioned above requires a thymic organ culture system and can be conducted in animals such as mice. However, it is difficult to apply this assay to humans because of the difficulty in obtaining human fetal thymus.

Therefore, a simplified method for determining the developmental potential of individual cells towards the myeloid, erythroid, and B cell lineages (MLP<MEB> assay) has been developed. This method uses a culture system that supports the development towards the myeloid, erythroid, and B cell lineages.

In this method, a sample containing hematopoietic stem and/or progenitor cells, such as murine fetal liver cells, murine fetal blood cells, murine bone marrow cells, or murine thymus cells may be examined. This method will also be used for the detection of human hematopoietic stem and/or progenitors in a sample such as cord blood, bone marrow, or peripheral blood.

A cell population in which hematopoietic stem and/or progenitor cells are enriched may be separated in the same way as in MLP<METB> assay. Individual cells contained in the population are cultured, and the type of cells produced in the culture is determined.

A culture system to be used in MLP<MEB> assay comprises stromal cells for differentiation into myeloid and B cells, and cytokines for differentiation into erythroid cells and also myeloid cells. Stromal cells for differentiation into myeloid and B cells include fibroblasts. Cytokines to be used include erythropoietin (Epo), interleukin 6 and granulocyte colony-stimulating factor (G-CSF). The amount of cytokines to be added to this culture system is preferably 2-5 U/ml for Epo, 2-10 ng/ml for IL-6 and 10-15 ng/ml for G-CSF.

Culturing may be carried out at 37° C. under the high oxygen submersion conditions (70% O₂, 25% N and 5% CO₂).

The cells produced in the culture are stained with antibodies specific for each cell lineage, and are analyzed with a flow cytometer, the fluorescence-activated cell sorter (FACS). From FACS analysis data, the type of each progenitor can be determined.

According to this simplified method of the present invention, it is possible to detect seven types of progenitors.

As shown in Example 2, single cells of Lin⁻ c-kit⁺ Sca-1⁺ group were cultured on a monolayer of cells of fibroblast cell line Tst-4 in the presence of Epo and G-CSF. As a result of analysis of the cells produced, progenitors which produce myeloid, erythroid, and B cells (M/E/B type) were detected. The frequency of M/E/B type progenitors among the cells of this group is almost the same as that of p-METB determined in the above-mentioned MLP<METB> assay. Therefore, this type of progenitors are thought to be the stem cells. Accordingly, the MLP<MEB> assay may be used for the detection of human stem and/or progenitor cells as a simplified method equivalent to MLP<METB> assay.

### EXAMPLES

The following examples are given to further illustrate this invention, and are not intended to limit the invention.

### Reference Example 1

The preparation of murine fetal liver cells, cell staining with various antibodies, analysis with a FACS Vantage, and separation of a progenitor-enriched population were carried out as follows.

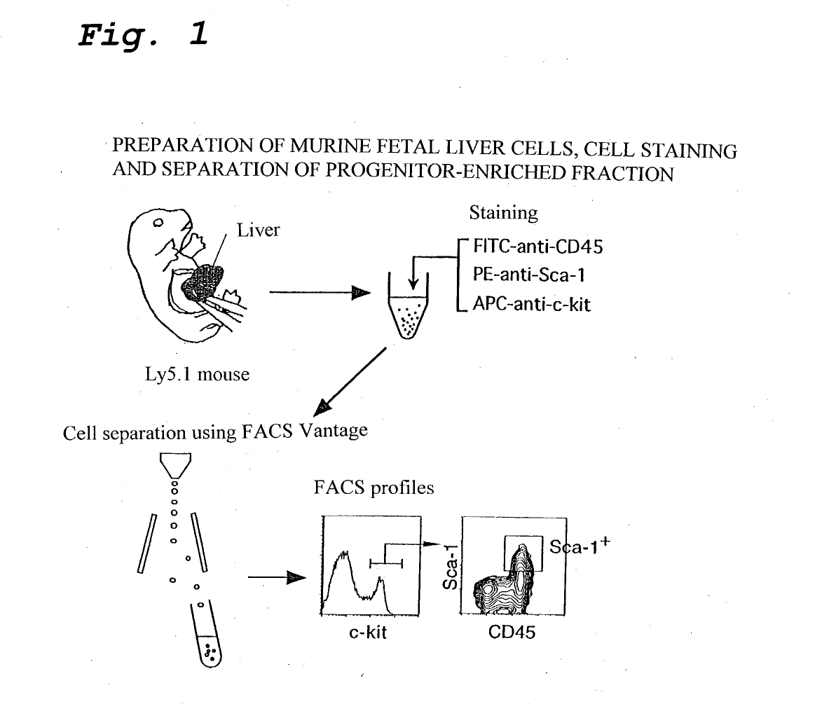

Fetal liver (FL) cells were taken from murine fetuses at 11-15 days post coitum (dpc) (C57BL/6Ly5.1 line) and a cell suspension was prepared. FL cells were fluorescently labeled with fluorescein (FITC)-labeled anti CD45 antibodies (FITC-anti-CD45), phycoerythrin (PE)-anti-Sca-1, and allophycocyanin (APC)-anti-c-kit (FIG. 1).

Staining patterns obtained from analysis with a FACS Vantage are as shown in FIG. 1. Hematopoietic stem and/or progenitor cells are enriched in the area enclosed by a rectangle at the bottom right of FIG. 1, i.e., in Sca-1⁺ group. Cells contained therein were examined for progenitor activity.

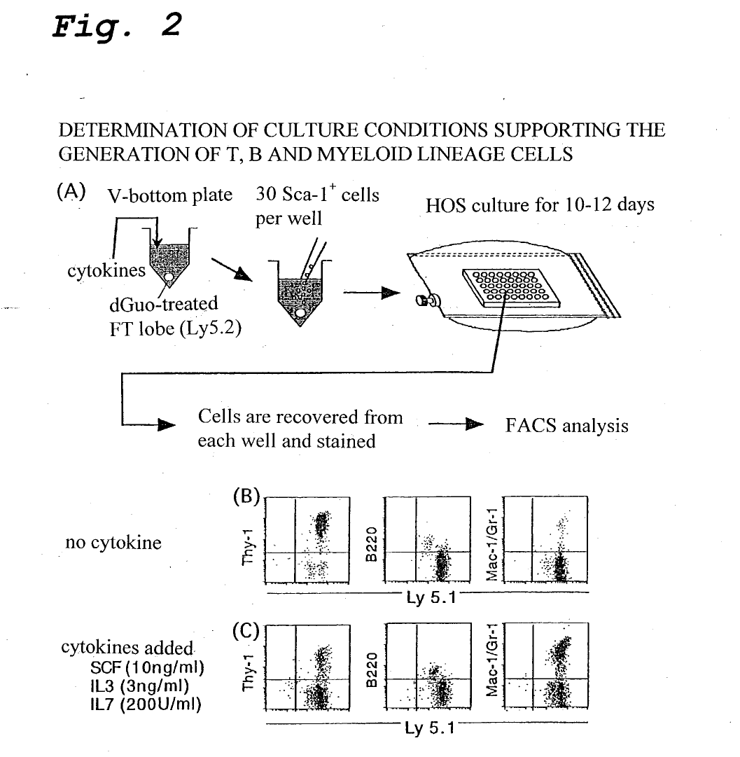

First, culture conditions allowing the development and growth of the myeloid, T and B cells were determined as shown in FIG. 2(A). Single dGuo-treated FT (C57BL/6Ly5.2 strain) lobes, which support T cell differentiation, were placed into the wells of a 96-well V-bottom plate, and cytokines were added to the culture medium to support the differentiation and growth of the myeloid and B cells. As shown in FIG. 2(A), 30 Sca-1⁺ murine fetal liver cells from 13 dpc fetuses of C57BL/6(ly5.1) mice were cultured together with a dGuo-treated FT lobe in a well of a 96-well plate in the presence of SCF (10 ng/ml), LI-3 (3 ng/ml) and LI-7 (5 ng/ml) under the HOS conditions (70% O₂, 5% CO₂, 25% N₂).

On day 12 of culture, the cells were harvested and divided into two cell batchs. Cells in each batch were stained with FITC-anti-Thy-1 (T cell marker), PE-anti-B220 (B cell marker), and APC-anti-Ly5.1 (donor cell marker), or with FITC-anti-Thy-1, PE-anti-Mac-1/Gr-1 (myeloid cell marker) and APC-anti-Ly5.1. The flow cytometric profiles of the cells are shown in FIGS. 2(B) and (C). The combination and concentration of cytokines shown in FIG. 2(C) represent the most effective culture condition for the MLP<MTB> assay.

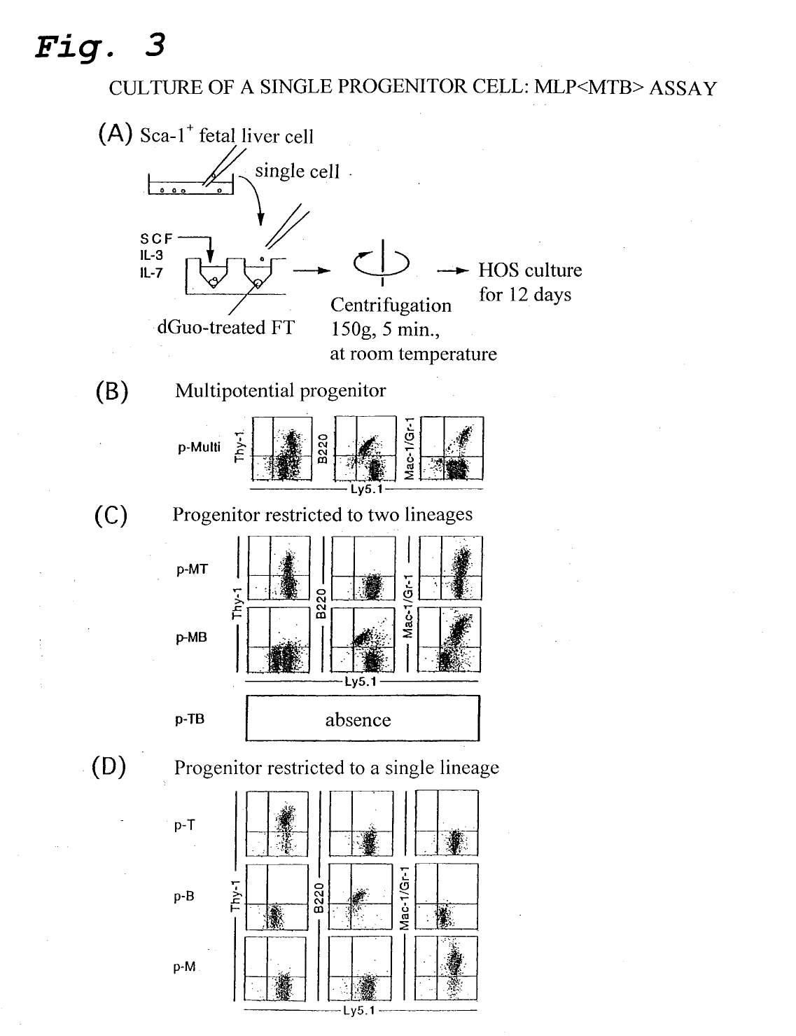

The MLP<MTB> assay comprises culturing individual progenitors under the above-mentioned conditions to detect the developmental potential of individual cells towards T, B and myeloid lineages. Single cells from Sca-1⁺ population of 13 dpc murine fetal liver were cultured as shown in FIG. 3(A). On day 12 of culture, the cells generated were recovered, stained and analyzed with a FACS Vantage in the same way as above. Six representative profiles of recovered cells are shown in FIG. 3(B)-(D). The developmental potential of each progenitor cell was judged from this FACS profile.

FIG. 3(B) shows that a single progenitor produced T, B, and myeloid cells (multipotent progenitor, p-Multi). FIG. 3(C) shows profiles of cells derived from a p-MT and a p-MB. p-MT produces T and myeloid cells but does not produce B cells at all. On the other hand, p-MB produces B and myeloid cells but does not produce T cells at all. The existence of p-MT and p-MB had never before been predicted.

Another possible one is p-TB which produces T and B cells and does not produce myeloid cells, but such a progenitor has never been detected at all, leading to the conclusion that p-TB does not exist.

FIG. 3(D) shows profiles of cells derived from a p-T, a p-B and a p-M, which produce T, B, and myeloid cells, respectively. It is demonstrated that p-T exists in fetal liver from normal as well as athymic nude mouse, and thus the commitment of hematopoietci stem cells to p-T is independent of the thymus.

The absence of p-TB indicates that p-T and p-B are produced from p-MT and p-MB, respectively.

### Example 1

Single Sca-1⁺ cells from 13 dpc murine fetal liver were cultured together with a dGuo-treated FT lobe in a well of a 96-well plate in the presence of SCF (10 ng/ml), LI-3 (3 ng/ml), LI-7 (5 ng/ml) and Epo (2 U/ml) under the HOS conditions (70% O₂, 5% CO₂, 25% N₂). Erythrocytes appear much earlier than cells of other lineages. On day 6 of culture, cells grown outside the lobe were harvested and assayed for erythrocytes. The recovered cells were stained with FITC-TER (erythrocyte marker) and PE-anti-Mac-1/Gr-1 and analyzed with a FACS Vantage. The lobe was cultured for additional 6 days, and the cells were recovered and assayed for T, B and myeloid cells. The cells were stained with various markers and analyzed with a FACS Vantage in the same way as mentioned in MLP<MTB> assay.

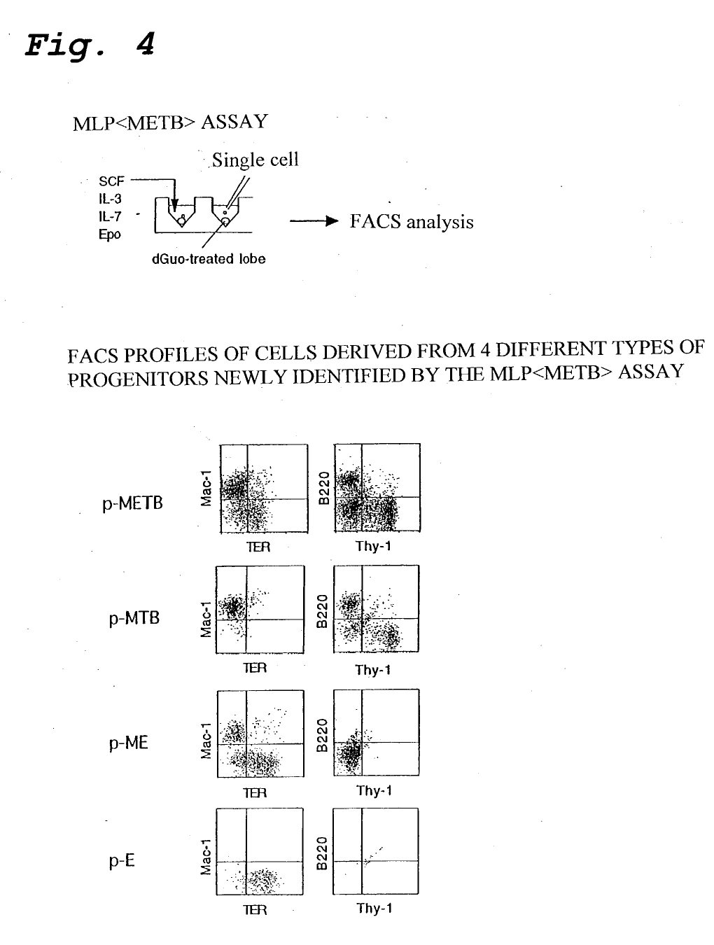

According to this assay, the p-Multi as determined by the MLP<MTB> assay, was found to comprise p-METB and p-MTB. Furthermore, p-ME and p-E were detected. Thus, nine types of progenitors were actually detected among theoretically possible 15 types. FIG. 4 shows FACS profiles of the cells derived from four types of progenitors, i.e., p-METB, p-MTB, p-ME, and p-E, which were not detected by the MLP<MTB> assay and were detected for the first time by the MLP<METB> assay. The p-METB is considered to be the stem cells. The p-MTB may represent common myelo-lymphoid progenitors, the presence of which was not expected prior to the present invention.

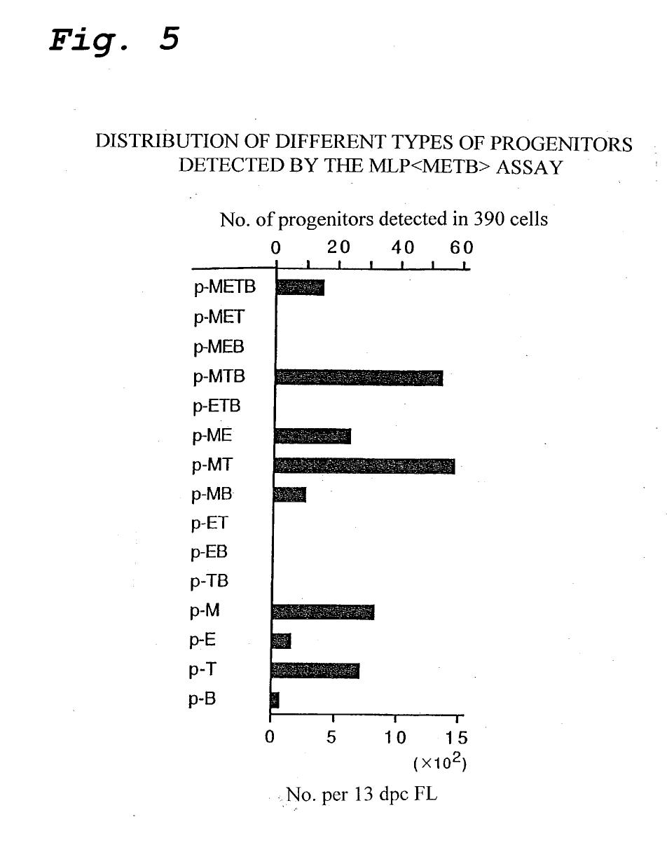

The results obtained by culturing and determining 390 Sca-1⁺ cells, and the distribution of different types of progenitors are shown in FIG. 5. Undetected types are p-MET, p-MEB, p-ETB, p-ET, p-EB, and p-TB.

### Example 2

With the MLP<MEB> assay, Sca-1⁺ cells from murine fetal liver were cultured individually and examined for their developmental potential.

Stromal cell line TSt-4 was monolayered in a 96-well flat bottom plate, and culture medium was supplemented with Epo (2 U/ml) and G-CSF (10 ng/ml). Single cells were seeded into each well. Grown cells were recovered on day 7 of culture, were stained with various markers, and were analyzed by a FACS Vantage in the same way as in Example 1. Six types of progenitors, M/E/B, M/E, M/B, M, E and B types, among theoretically possible seven types were detected with this method. E/B type has never been detected, therefore this type is thought to be absent. M/E/B type progenitors detected in this method are considered to be hematopoietic stem cells.

According to the present invention, progenitors in various steps of hematopoiesis process can be distinguished. This makes it possible for the first time to elucidate the hematopoiesis process covering myelo-erythroid and T/B lymphoid lineages. Also, MLP<METB> assay of the present invention enables us to detect in vitro the hematopoietic stem cells, which has been impossible prior to the present invention, and MLP<MEB> assay of the present invention can be applied to the detection of human hematopoietic stem cells. This method is useful in a variety of therapeutic purposes in humans. For example, hematopoietic injuries due to congenital or artificial gene deficiency can be assayed definitely, and it will become possible to use appropriate types of progenitor cells in gene therapy.

Thus, the present invention can be expected to greatly contribute to studies of differentiation of hematopoietic cells including lymphocyte lineages and to medical treatments.

It will be apparent to one of ordinary skill in the art that many changes and modifications can be made in the invention without departing from the spirit or scope of the appended claims.

What is claimed is:

1. A method for detection of hematopoietic stem cells and/or hematopoietic progenitor cells, comprising the steps of culturing individual cells in a culture environment that supports the generation of myeloid, erythroid, T and B lineage cells and determining the developmental potential of individual cells towards these four lineages.

2. A method according to claim 1, wherein the determination of the developmental potential is made by analyzing the cells generated from a single progenitor using a flow cytometer.

3. A culture system for use in detecting hematopoietic stem cells and/or hematopoietic progenitor cells, which supports the generation of myeloid, erythroid, T and B lineage cells.

4. A culture system according to claim 3, which comprises a thymic organ culture system and further comprises cytokines that support the growth of myeloid, erythroid, and B lineage cells.

5. A method for detection of hematopoietic stem cells and/or hematopoietic progenitor cells, comprising the steps of culturing individual cells in a culture environment that supports the generation of myeloid, erythroid and B lineage cells and determining the developmental potential of individual cells towards these three lineages.

6. A method according to claim 5, wherein the assay for developmental potential is made by analyzing the cells generated from a single progenitor using a flow cytometer.

7. A culture system for use in detecting hematopoietic stem cells and/or hematopoietic progenitor cells, which supports the generation of myeloid, erythroid and B lineage cells.

8. A culture system according to claim 7, which comprises stromal cells that support the development towards the myeloid and B cell lineage, and further comprises cytokines that support the generation of erythroid cells.
